(19) United States
(12) Patent Application Publication
(10) Pub. No.: US 2020/0221962 A1
(43) Pub. Date: Jul. 16, 2020

(54) MEASUREMENT DEVICE AND MEASUREMENT METHOD

(71) Applicants: OMRON Corporation, Kyoto (JP); OMRON HEALTHCARE Co., Ltd., Kyoto (JP)

(72) Inventor: Naoki MATSUMOTO, Kyoto (JP)

(21) Appl. No.: 16/748,888

(22) Filed: Jan. 22, 2020

Related U.S. Application Data
(63) Continuation of application No. PCT/JP2018/022026, filed on Jun. 8, 2018.

(30) Foreign Application Priority Data
Jul. 26, 2017 (JP) 2017-144381

(51) Int. Cl. A61B 5/021; A61B 5/04; A61B 5/0404; A61B 5/044; A61B 5/00

(52) U.S. Cl. CPC A61B 5/02125; A61B 5/04014; A61B 5/681; A61B 5/044; A61B 5/0404

(57) ABSTRACT

A measurement device (1) performs filtering processing via an analog filter (403) with a predetermined transfer function on a first signal indicating a pulse wave of a subject and a second signal indicating a pulse wave or an electrocardiogram of the subject; performs filtering processing via a digital filter with the predetermined transfer function on third time series data, which is first time series data of the first signal arranged in reverse chronological order, and fourth time series data, which is second time series data of the second signal arranged in reverse chronological order; and calculates a pulse transit time on the basis of a signal indicated by fifth time series data, which is the third time series data post filtering processing arranged in chronological order, and a signal indicated by sixth time series data, which is the fourth time series data post filtering processing arranged in chronological order.

### CROSS-REFERENCE TO RELATED APPLICATIONS

This is a continuation of International Application PCT/JP2018/022026, with an international filing date of Jun. 8, 2018, filed by applicant, the disclosure of which is hereby incorporated by reference in its entirety.

### TECHNICAL FIELD

The present disclosure relates to a measurement device and a measurement method and particularly relates to a measurement device and a measurement method for measuring pulse transit time.

### BACKGROUND ART

A method for measuring the propagation time of pulse waves propagating through an artery (pulse transit time (PTT)) is known. For example, WO 2014/132713 (Patent Document 1) describes a pulse transit time measurement device. The pulse transit time measurement device detects a peak of an electrocardiographic signal on which signal processing including filtering processing has been performed and a peak of a photoelectric pulse wave signal on which signal processing including filtering processing has been performed, corrects the peak of the electrocardiographic signal and the peak of the photoelectric pulse wave signal on the basis of a delay time of the electrocardiographic signal and a delay time of the photoelectric pulse wave signal, and calculates a pulse transit time from a time difference between the peak of the photoelectric pulse wave signal and the peak of the electrocardiographic signal which have been corrected.

### CITATION LIST

### Patent Literature

Patent Document 1: WO 2014/132713

### SUMMARY OF INVENTION

### Technical Problem

Patent Document 1 describes a method of detecting a peak of an electrocardiographic signal and a peak of a photoelectric pulse wave signal with high precision by which a pulse transit time can be determined with high precision. Specifically, the pulse transit time measurement device according to Patent Document 1 analyses a frequency component of the electrocardiographic signal and a frequency component of the photoelectric pulse wave signal, calculates a delay time of the electrocardiographic signal and a delay time of the photoelectric pulse wave signal using a table in which relationships between frequency components and delay times (amount of peak shift) are defined, and calculates a pulse transit time from a time difference between the peak of the photoelectric pulse wave signal and the peak of the electrocardiographic signal which have been corrected on the basis of the delay time.

However, according to the method described in Patent Document 1, a database must be prepared in advance. Also, any errors in the database may affect calculations, producing an error in the delay time and thus in the pulse transit time.

An object of an embodiment of the present disclosure is to provide a measurement device and a measurement method capable of measuring pulse transit time easily and with high precision.

### Solution to Problem

A measurement device according to an embodiment includes:

a first sensor that detects a first signal indicating a pulse wave of a subject;

a second sensor that detects a second signal indicating a pulse wave or an electrocardiogram of the subject;

a first signal processing unit that performs filtering processing via an analog filter with a predetermined transfer function on the first signal detected by the first sensor and the second signal detected by the second sensor and converts the first signal detected by the first sensor and the second signal detected by the second sensor to digital data; and

a second signal processing unit that performs signal processing on first time series data of the first signal converted to digital data by the first signal processing unit and second time series data of the second signal converted to digital data by the first processing unit. The second signal processing unit

generates third time series data, the third time series data being the first time series data arranged in reverse chronological order,

generates fourth time series data, the fourth time series data being the second time series data arranged in reverse chronological order,

performs filtering processing via a digital filter with the predetermined transfer function on the third time series data and the fourth time series data,

generates fifth time series data, the fifth time series data being the third time series data post filtering processing by the digital filter arranged in chronological order, and

generates sixth time series data, the sixth time series data being the fourth time series data post filtering processing by the digital filter arranged in chronological order. The measurement device further includes a time calculation unit that calculates a pulse transit time on the basis of a signal indicated by the fifth time series data and a signal indicated by the sixth time series data.

Preferably, the second signal is a signal indicating a pulse wave. Also, the first sensor and the second sensor each detect a pulse wave at a portion of an artery running through a target measurement site of the subject corresponding to where the first sensor and the second sensor are located.

Preferably, the time calculation unit calculates a time difference between a rising time point of the signal indicated by the fifth time series data and a rising time point of the signal indicated by the sixth time series data as a pulse transit time, or

calculates a time difference between a peak time point of the signal indicated by the fifth time series data and a peak time point of the signal indicated by the sixth time series data as a pulse transit time.

Preferably, the second signal is a signal indicating an electrocardiogram. Also, the time calculation unit calculates a time difference between a rising time point of the signal indicated by the fifth time series data and a peak time point of the signal indicated by the sixth time series data as a pulse transit time.

Preferably, the measurement device further includes a data storage unit for storing the first time series data and the second time series data. Also, the second signal processing unit executes the signal processing when the first time series data and the second time series data for a predetermined amount of time are accumulated in the data storage unit.

Preferably, the measurement device further includes a blood pressure calculation unit that calculates a blood pressure value on the basis of a pulse transit time calculated by the time calculation unit.

Preferably, the measurement device further includes a display, and a display control unit that displays a blood pressure value calculated by the blood pressure calculation unit on the display.

A measurement method according to another embodiment includes:

detecting a first signal indicating a pulse wave of a subject;

detecting a second signal indicating a pulse wave or an electrocardiogram of the subject;

performing filtering processing via an analog filter with a predetermined transfer function on the first signal and the second signal and converting the first signal and the second signal to digital data;

generating third time series data, the third time series data being the first time series data of the first signal converted to digital data arranged in reverse chronological order;

generating fourth time series data, the fourth time series data being the second time series data of the second signal converted to digital data arranged in reverse chronological order;

performing filtering processing via a digital filter with the predetermined transfer function on the third time series data and the fourth time series data;

generating fifth time series data, the fifth time series data being the third time series data post filtering processing by the digital filter arranged in chronological order;

generating sixth time series data, the sixth time series data being the fourth time series data post filtering processing by the digital filter arranged in chronological order; and

calculating a pulse transit time on the basis of a signal indicated by the fifth time series data and a signal indicated by the sixth time series data.

### Advantageous Effects of Invention

According to the present disclosure, a pulse transit time can be easily measured with high precision.

### BRIEF DESCRIPTION OF DRAWINGS

FIG. 1 is a perspective view of the appearance of a blood pressure monitor.

FIG. 2 is a diagram schematically illustrating a cross-section of a left wrist taken perpendicular to the longitudinal direction, with a blood pressure monitor being worn on the left wrist.

FIG. 3 is a diagram illustrating a flat layout of an electrode group for measuring impedance, with a blood pressure monitor worn on a left wrist.

FIG. 4 is a block diagram illustrating the hardware configuration of a control system of a blood pressure monitor.

FIGS. 5A and 5B are schematic diagrams for describing blood pressure measurement on the basis of pulse transit time.

FIG. 6 is a schematic cross-sectional view along the longitudinal direction of a wrist of a blood pressure monitor being worn on a left wrist when performing blood pressure measurement via the oscillometric method.

FIGS. 7A to 7C are diagrams for describing the need for an analog filter.

FIG. 8 is a diagram for describing the phase characteristics of a filter.

FIG. 9 is a block diagram illustrating the functional configuration of a blood pressure monitor.

FIG. 10 is a diagram for explaining the advantages of digital signal processing according to the present embodiment.

FIG. 11 is a flowchart illustrating a processing procedure of measuring a blood pressure value on the basis of pulse transit time.

### DESCRIPTION OF EMBODIMENTS

Embodiments of the present invention will be described below with reference to the drawings. In the following description, like components are given like numerals. Names and functions thereof are also the same. Thus, the detailed description of such components is not repeated.

In the following, a blood pressure monitor will be used as an example of a “measurement device” for measuring pulse transit time. However, the measurement device is not limited to a blood pressure monitor and may be any device that includes a sensor that detects a pulse wave signal (or an electrocardiographic signal) and a processing device that processes the signal detected by the sensor.

### Configuration of Blood Pressure Monitor

### Appearance and Cross-Sectional Configuration

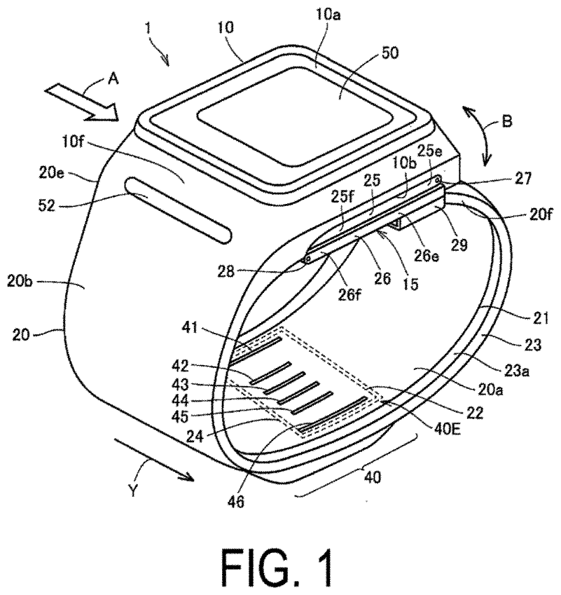

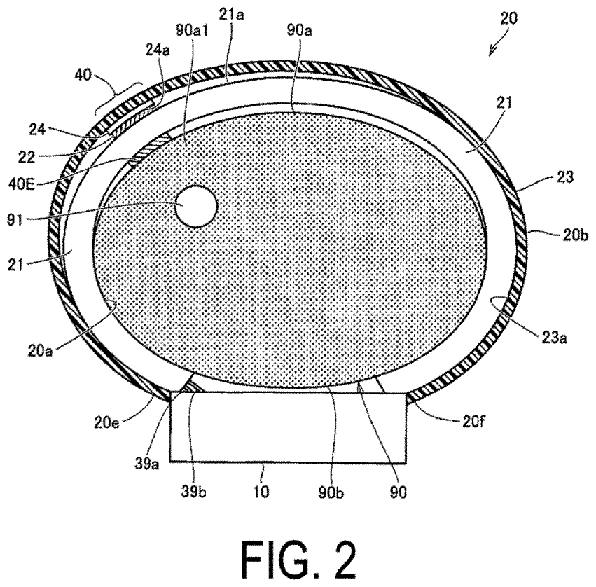

FIG. 1 is a perspective view of the appearance of a blood pressure monitor 1. FIG. 2 is a diagram schematically illustrating a cross section of a left wrist 90 taken perpendicular to the longitudinal direction, with the blood pressure monitor 1 being worn on the left wrist 90 (also referred to as the “worn state” below). In the present embodiment, the left wrist 90 is the target measurement site. Note that the “target measurement site” measured by the blood pressure monitor 1 is only required to be a site through which an artery runs. The target measurement site may be, for example, an upper limb, such as the wrist or the upper arm, or a lower limb, such as the ankle or the upper thigh.

Referring to FIGS. 1 and 2, a belt 20 is an elongate band-like member that is worn around the left wrist 90 in the circumferential direction. The dimensions (width dimension) of the belt 20 in a width direction Y is approximately 30 mm, for example. The belt 20 includes a band 23 including an outer circumferential surface 20b and a compression cuff 21.

The compression cuff 21 is attached along an inner circumferential surface 23a of the band 23 and includes an inner circumferential surface 20a that comes into contact with the left wrist 90. The compression cuff 21 is configured as a fluid bag and includes two stretchable polyurethane sheets layered in the thickness direction that are fused along the edge portions. The fluid bag is only required to be a bag-like member configured to accommodate a fluid. “Fluid” includes both liquids and gases, such as water and air.

A body 10 is integrally formed with an end portion 20e on one end of the belt 20. Note that the belt 20 and the body 10 may be formed separately or may have an integral configuration in which the body 10 is attached to the belt 20 via an engagement member (for example, a hinge). In the present embodiment, in the worn state, the portion where the body 10 is disposed corresponds to a back side surface (surface on the side of the back of the hand) 90b of the left wrist 90 (see FIG. 2). In FIG. 2, a radial artery 91 is illustrated running through the left wrist 90 near the palm side surface (surface on the side of the palm of the hand) 90a.

As illustrated in FIG. 1, the body 10 has a three-dimensional shape having thickness in the direction perpendicular to the outer circumferential surface 20b of the belt 20. The body 10 is formed small and thin so as not to interfere with the daily activity of the subject (user). The body 10 has a truncated quadrangular pyramid profile protruding outward from the belt 20.

A display 50 is provided on a top surface (the surface furthest from the target measurement site) 10a of the body 10. An operation portion 52 for a user to input an instruction is provided along a side surface (a side surface on the front side of the left hand in FIG. 1) 10f of the body 10.

An impedance measurement portion 40 is provided on the inner circumferential surface 20a of the belt 20 (i.e., the inner circumferential surface 20a of the compression cuff 21) at a portion between the end portion 20e and an end portion 20f at either ends of the belt 20.

An electrode group 40E is disposed on the inner circumferential surface 20a at the portion where the impedance measurement portion 40 is disposed. The electrode group 40E includes six plate-like (or sheet-like) electrodes 41 to 46 disposed separated from one another in the width direction Y of the belt 20. In the worn state, the location where the electrode group 40E is located corresponds to the radial artery 91 of the left wrist 90.

A solid material 22 is disposed on an outer circumferential surface 21a opposite the inner circumferential surface 20a at a position corresponding to the electrode group 40E. A pressing cuff 24 is disposed on the outer circumferential side of the solid material 22. The pressing cuff 24 is an expandable member that locally suppresses a region corresponding to the electrode group 40E with respect to the circumferential direction of the compression cuff 21. The pressing cuff 24 is disposed on the inner circumferential surface 23a (the surface on the side closer to the left wrist 90) of the band 23 constituting the belt 20 (see FIG. 2). The band 23 is made of a plastic material that is flexible in the thickness direction and that is non-stretchable in the circumferential direction (longitudinal direction).

The pressing cuff 24 is a fluid bag that stretches in the thickness direction of the belt 20. Specifically, the pressing cuff 24 is worn around the left wrist 90. When fluid is supplied, the pressing cuff 24 enters a pressurized state, and when fluid is discharged, the pressing cuff 24 enters a non-pressurized state. The pressing cuff 24, for example, is configured as a fluid bag and includes two stretchable polyurethane sheets layered in the thickness direction that are fused along the edge portions.

The solid material 22 is disposed on an inner circumferential surface 24a of the pressing cuff 24 (on the side closer to the left wrist 90) at a portion corresponding to the electrode group 40E. The solid material 22 is made of, for example, a resin (for example, polypropylene) shaped like a plate having a thickness of approximately from 1 to 2 mm. In the present embodiment, the belt 20, the pressing cuff 24, and the solid material 22 are used as a pressing portion.

As illustrated in FIG. 1, a bottom surface (a surface on the side closest to the target measurement site) 10b of the body 10 and the end portion 20f of the belt 20 are connected via a tri-fold buckle 15 (also simply referred to as “buckle 15” below).

The buckle 15 includes a plate-like member 25 disposed on the outer circumferential side and a plate-like member 26 disposed on the inner circumferential side. A first end portion 25e of the plate-like member 25 is pivotably mounted to the body 10 via a connecting rod 27 that extends in the width direction Y. A second end portion 25f of the plate-like member 25 is pivotably mounted to a first end portion 26e of the plate-like member 26 via a connecting rod 28 that extends in the width direction Y. A second end portion 26f of the plate-like member 26 is fixed to the belt 20 near the end portion 20f via a fixing portion 29.

The position where the fixing portion 29 is attached in respect to the circumferential direction of the belt 20 is changeable and set in advance to match the circumference of the left wrist 90 of the user. In this way, the blood pressure monitor 1 (belt 20) has an overall roughly annular shape and has a configuration in which the bottom surface 10b of the body 10 and the end portion 20f of the belt 20 can open and close via the buckle 15 in the direction of arrow B in FIG. 1.

When the user puts on the blood pressure monitor 1 on the left wrist 90, the user opens the buckle 15 to increase the diameter of the circle made by the belt 20 and puts the left hand through the belt 20 from the direction of arrow A in FIG. 1. Next, as illustrated in FIG. 2, the user adjusts the angular position of the belt 20 around the left wrist 90 to position the impedance measurement portion 40 of the belt 20 above the radial artery 91 running through the left wrist 90. In this way, the electrode group 40E of the impedance measurement portion 40 comes into contact with a portion 90a1 of the palm side surface 90a of the left wrist 90 corresponding to the radial artery 91. In this state, the user closes and fixes the buckle 15. In this manner, the user puts the blood pressure monitor 1 (belt 20) on the left wrist 90.

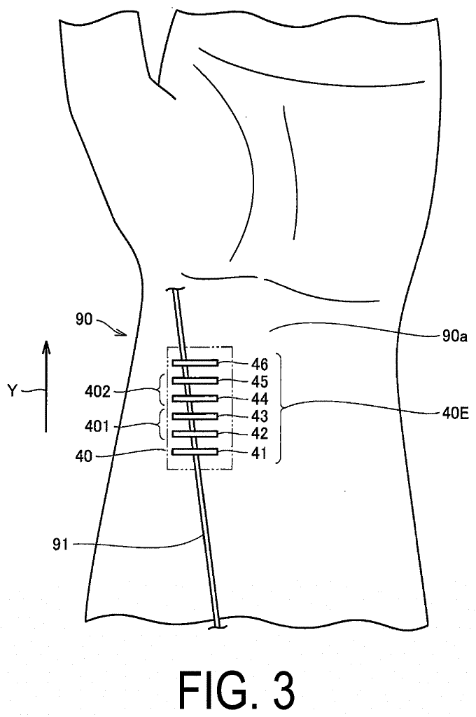

FIG. 3 is a diagram illustrating a flat layout of an electrode group for measuring impedance, with the blood pressure monitor 1 worn on the left wrist 90. Referring to FIG. 3, in the worn state, the electrode group 40E of the impedance measurement portion 40 are arranged side by side in the longitudinal direction of the wrist, corresponding to the location of the radial artery 91 of the left wrist 90. The electrode group 40E includes the pair of current electrodes 41, 46 disposed on either side and the pair of detection electrodes 42, 43 and the pair of detection electrodes 44, 45 disposed between the current electrodes 41, 46. A pulse wave sensor 401 includes the pair of detection electrode 42, 43 and a pulse wave sensor 402 includes the pair of detection electrodes 44, 45.

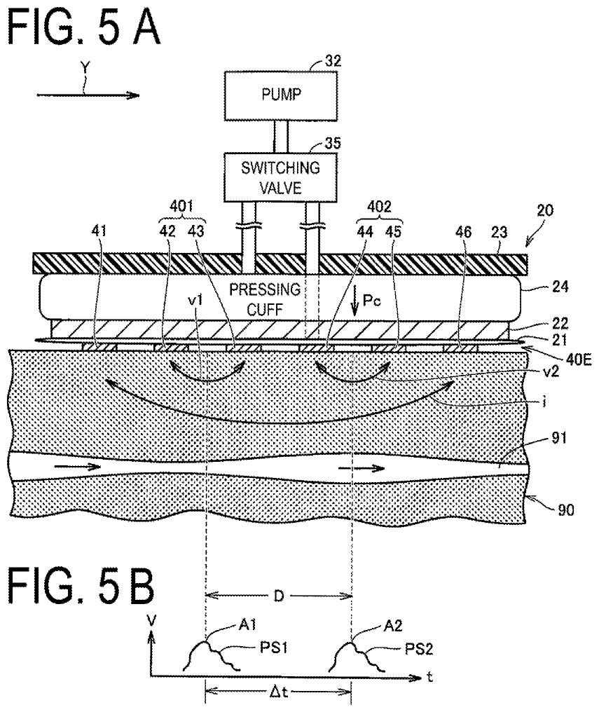

The pair of detection electrodes 44, 45 are disposed downstream, in terms of the blood flow of the radial artery 91, of the pair of detection electrodes 42, 43. A distance D in the width direction Y between a central point between the pair of detection electrodes 42, 43 and a central point between the pair of detection electrodes 44, 45 (see FIG. 5A below) is 20 mm, for example. The distance D corresponds to the space between the pulse wave sensor 401 and the pulse wave sensor 402. Also, the space in the width direction Y between the pair of detection electrodes 42, 43 and the space in the width direction Y between the pair of detection electrodes 44, 45 are 20 mm, for example.

The electrode group 40E having a flat configuration such as that described allows the belt 20 of the blood pressure monitor 1 to have a thin configuration. Also, because the electrode group 40E has a flexible configuration, the compression of the left wrist 90 by the compression cuff 21 is not disrupted and the precision of blood pressure measurement performed via the oscillometric method described below is not decreased.

### Hardware Configuration

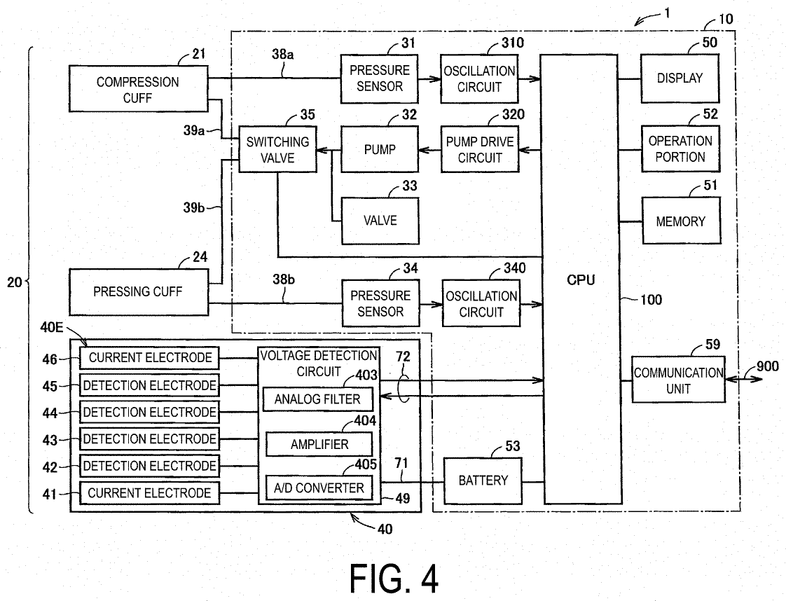

FIG. 4 is a block diagram illustrating the hardware configuration of a control system of the blood pressure monitor 1. Referring to FIG. 4, the body 10 includes a central processing unit (CPU) 100 that functions as a control unit; the display 50; a memory 51 functioning as a storage unit; the operation portion 52; a battery 53; and a communication unit 59. The body 10 also includes a pressure sensor 31, a pump 32, a valve 33, a pressure sensor 34, and a switching valve 35. The switching valve 35 switches the connection destination of the pump 32 and the valve 33 to the compression cuff 21 or the pressing cuff 24.

Furthermore, the body 10 includes an oscillation circuit 310 and an oscillation circuit 340 that convert the output from the pressure sensor 31 and the pressure sensor 34 into a frequency; and a pump drive circuit 320 that drives the pump 32. The impedance measurement portion 40 includes the electrode group 40E and a voltage detection circuit 49.

The display 50 is constituted by, for example, an organic electro luminescence (EL) display and displays information relating to blood pressure measurement such as blood pressure measurement results and other information, in accordance with a control signal from CPU 100. Note that the display 50 is not limited to being constituted by an organic EL display and may be constituted by another type of display such as a liquid crystal display (LCD) or another type of display, for example.

The operation portion 52 is constituted by, for example, a push type switch and inputs to the CPU 100 an operation signal in response to an instruction from the user to start or stop blood pressure measurement. Note that the operation portion 52 is not limited to being a push type switch and may be, for example, a pressure sensitive type (resistance type) or a proximity type (capacitance type) touch panel type switch. The body 10 also includes a microphone (not illustrated) and may receive an instruction to start blood pressure measurement from the user via sound.

The memory 51 non-transitorily stores data of a program for controlling the blood pressure monitor 1, data used to control the blood pressure monitor 1, settings data for setting various functions of the blood pressure monitor 1, data of blood pressure values of measurement results, and the like. The memory 51 is used as a working memory or the like for when a program is executed.

The CPU 100 executes various functions as a control unit in accordance with a program for controlling the blood pressure monitor 1 stored in the memory 51. For example, when performing blood pressure measurements via the oscillometric method, the CPU 100 performs control to drive the pump 32 (and the valve 33) on the basis of a signal from the pressure sensor 31 in response to an instruction to start blood pressure measurement from operation portion 52. Additionally, the CPU 100 performs control to calculate the blood pressure value on the basis of a signal from the pressure sensor 31.

When blood pressure measurement is executed on the basis of pulse transit time, the CPU 100 performs control to drive the valve 33 for discharging the air inside the compression cuff 21 in response to an instruction to start blood pressure measurement from the operation portion 52. Also, the CPU 100 performs control to drive the switching valve 35 and switch the connection destination of the pump 32 (valve 33) to the pressing cuff 24. Furthermore, the CPU 100 performs control to calculate the blood pressure value on the basis of a signal from the pressure sensor 34.

The communication unit 59 is controlled by the CPU 100 and sends predetermined information to an outside device via a network 900, receives information from an outside device via the network 900 and relays the information to the CPU 100, and the like. Communication via the network 900 may be wireless or wired. In this example, the network 900 is the Internet, but is not limited thereto and may be other types of networks such as a local area network (LAN) or may be a one-to-one communication using a USB cable or the like. The communication unit 59 may include a micro USB connector.

The pump 32 and the valve 33 are connected to the compression cuff 21 and the pressing cuff 24 via the switching valve 35 and air lines 39a, 39b. The pressure sensor 31 is connected to the compression cuff 21 via the air line 38a and the pressure sensor 34 is connected to the pressing cuff 24 via the air line 38b. The pressure sensor 31 detects the pressure in the compression cuff 21 via the air line 38a. The switching valve 35 is driven on the basis of a control signal from the CPU 100 and switches the connection destination of the pump 32 and the valve 33 to the compression cuff 21 or the pressing cuff 24.

The pump 32 is constituted by a piezoelectric pump, for example. When the connection destination of the pump 32 and the valve 33 is switched to the compression cuff 21 via the switching valve 35, the pump 32 supplies air, i.e., pressurizing fluid, to the compression cuff 21 via an air line 39a to raise the pressure (cuff pressure) in the compression cuff 21. When the connection destination of the pump 32 and the valve 33 is switched to the pressing cuff 24 via the switching valve 35, the pump 32 supplies air, i.e., pressurizing fluid, to the pressing cuff 24 via an air line 39b to raise the pressure (cuff pressure) in the pressing cuff 24.

The pump 32 is provided with the valve 33, and the valve 33 is configured to be controlled to be open and closed to correspond with the pump 32 being on and off. Specifically, when the connection destination of the pump 32 and the valve 33 is switched to the compression cuff 21 via the switching valve 35 and the pump 32 is turned on, the valve 33 closes and air fills the inside of the compression cuff 21. When the pump 32 is turned off, the valve 33 opens and the air in the compression cuff 21 is discharged to the atmosphere via the air line 39a.

When the connection destination of the pump 32 and the valve 33 is switched to the pressing cuff 24 via the switching valve 35 and the pump 32 is turned on, the valve 33 closes and air fills the inside of the pressing cuff 24. When the pump 32 is turned off, the valve 33 opens and the air in the pressing cuff 24 is discharged to the atmosphere via the air line 39b. The valve 33 functions as a check valve, preventing the discharged air from flowing in reverse. The pump drive circuit 320 drives the pump 32 on the basis of a control signal from the CPU 100.

The pressure sensor 31, for example, is a piezoresistive pressure sensor and is connected to the pump 32, the valve 33, and the compression cuff 21 via the air line 38a. The pressure sensor 31 detects the pressure of the belt 20 (compression cuff 21) via the air line 38a using atmospheric pressure as a reference (zero), for example, and outputs a time series signal.

The oscillation circuit 310 outputs to the CPU 100 a frequency signal having a frequency corresponding to an electrical signal based on the change in electric resistance of the pressure sensor 31 due to the piezoresistive effect. The output of the pressure sensor 31 is used to control the pressure of the compression cuff 21 and to calculate blood pressure values (including for systolic blood pressure (SBP) and for diastolic blood pressure (DBP)) via the oscillometric method.

The pressure sensor 34, for example, is a piezoresistive pressure sensor and is connected to the pump 32, the valve 33, and the pressing cuff 24 via the air line 38b. The pressure sensor 34 detects the pressure of the pressing cuff 24 via the air line 38b using atmospheric pressure as a reference (zero), for example, and outputs a time series signal.

The oscillation circuit 340 oscillates corresponding to an electrical signal based on the change in electric resistance of the pressure sensor 34 due to the piezoresistive effect and outputs to the CPU 100 a frequency signal having a frequency corresponding to the electrical signal of the pressure sensor 34. The output of the pressure sensor 34 is used to control the pressure of the pressing cuff 24 and to calculate the blood pressure on the basis of pulse transit time. When the pressure of the pressing cuff 24 is controlled to measure the blood pressure on the basis of pulse transit time, the CPU 100 controls the pump 32 and the valve 33 and reduces the pressure, i.e., cuff pressure, in accordance with various conditions.

The battery 53 supplies power to the various elements the body 10 is provided with. The battery 53 supplies power to the voltage detection circuit 49 of the impedance measurement portion 40 via a wire 71. The wire 71 is disposed together with a wire 72 for signals between the band 23 and the compression cuff 21 of the belt 20 and extends in the circumferential direction of the belt 20 between the body 10 and the impedance measurement portion 40.

The voltage detection circuit 49 of the impedance measurement portion 40 operates in accordance with an instruction from the CPU 100. Specifically, the voltage detection circuit 49 includes an analog filter 403, an amplifier 404, and an analog/digital (A/D) converter 405. The voltage detection circuit 49 may further include a booster circuit that boosts the power supply voltage and a voltage adjustment circuit that adjusts the boosted voltage to a predetermined voltage.

### Summary of Blood Pressure Measurement on the Basis of Pulse Transit Time

FIGS. 5A and 5B are schematic diagrams for describing blood pressure measurement on the basis of pulse transit time. Specifically, FIG. 5A is a schematic cross-sectional view along the longitudinal direction of the wrist when performing blood pressure measurement on the basis of pulse transit time with the blood pressure monitor 1 worn on the left wrist 90. FIG. 5B illustrates the waveforms of pulse wave signals PS1, PS2.

Referring to FIG. 5A, a high frequency constant current i with a frequency of 50 kHz and a current value of 1 mA, for example, flows when the voltage detection circuit 49 applies a predetermined voltage across the pair of current electrodes 41, 46 using a booster circuit, a voltage adjustment circuit, or the like.

The voltage detection circuit 49 detects a voltage signal v1 between the pair of detection electrodes 42, 43 of the pulse wave sensor 401 and a voltage signal v2 between the detection electrodes 44, 45 of the pulse wave sensor 402. Specifically, the voltage detection circuit 49 receives an input of the voltage signal v1 detected by the pulse wave sensor 401 and an input of the voltage signal v2 detected by the pulse wave sensor 402. The voltage signals v1, v2 are signals that indicate pulse waves in the subject. Specifically, the voltage signals v1, v2 represent a change in electrical impedance caused by a pulse wave of the blood flow of the radial artery 91 at the portion corresponding to the where the pulse wave sensors 401, 402 are located on the palm side surface 90a of the left wrist 90.

The analog filter 403 of the voltage detection circuit 49 has a transfer function G and performs filtering processing on the amplified voltage signals v1, v2. Specifically, the analog filter 403 removes noise outside of the frequency characterizing the voltage signals v1, v2 (pulse wave signals) and performs filtering processing to improve the S/N ratio. The amplifier 404 is constituted by, for example, by an op-amp or the like and amplifies the filtered voltage signals v1, v2. The A/D converter 405 converts the amplified voltage signals v1, v2 from analog data to digital data and outputs it to the CPU 100 via the wire 72.

The CPU 100 performs a predetermined signal processing on the input voltage signals v1, v2 (digital data) and generates pulse wave signals PS1, PS2 having a mountain-like waveform as illustrated in FIG. 5B. The prescribed signal processing will be described in detail below.

Note that the voltage signals yl, v2 are approximately 1 mv, for example. Also, peaks A1, A2 of the pulse wave signals PS1, PS2 are approximately 1 V, for example. In the case where the pulse wave velocity (PWV) of the blood flow of the radial artery 91 ranges from 1000 cm/s to 2000 cm/s, a time difference Δt between the pulse wave signal PS1 and the pulse wave signal PS2 ranges from 1.0 ms to 2.0 ms, where the distance D between the pulse wave sensor 401 and the pulse wave sensor 402 is 20 mm.

As illustrated in FIG. 5A, the pressing cuff 24 is in a pressurized state, and the compression cuff 21 is in a non-pressurized state with air being discharged from inside the compression cuff 21. The pressing cuff 24 and the solid material 22 are disposed, with respect to the artery direction of the radial artery 91, across the pulse wave sensor 401, the pulse wave sensor 402, and the pair of current electrodes 41, 46. As such, when the pressing cuff 24 is pressurized by the pump 32, the pulse wave sensor 401, the pulse wave sensor 402, and the pair of current electrodes 41, 46 are pressed against the palm side surface 90a of the left wrist 90 by the solid material 22.

The pressing force against the palm side surface 90a of the left wrist 90 of each of the pair of current electrodes 41, 46, the pulse wave sensor 401, and the pulse wave sensor 402 can be set to an appropriate value. In the present embodiment, the pressing cuff 24, which is a fluid bag, is used as the pressing portion. This allows the pump 32 and the valve 33 to be used together with the compression cuff 21 and allows the configuration to be simplified. Also, the pulse wave sensor 401, the pulse wave sensor 402, and the pair of current electrodes 41, 46 can be pressed by the solid material 22. This allows the pressing force against the target measurement site to be even and blood pressure measurement on the basis of pulse transit time to be performed with high precision.

### Summary of Blood Pressure Measurement Via the Oscillometric Method

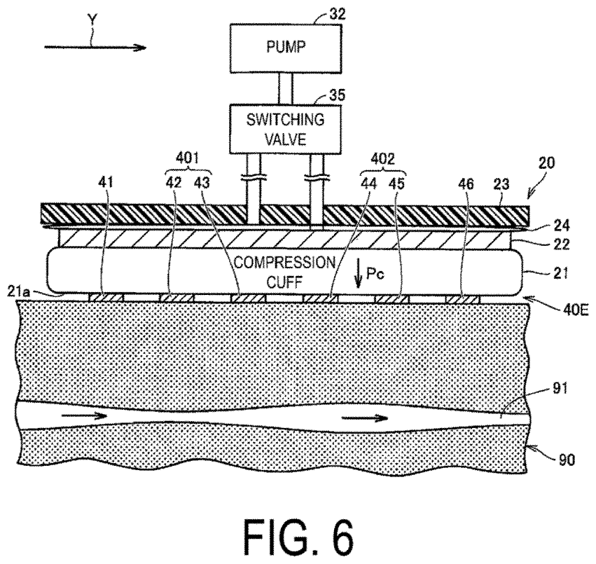

FIG. 6 is a schematic cross-sectional view along the longitudinal direction of the wrist of the blood pressure monitor 1 being worn on the left wrist 90 when performing blood pressure measurement via the oscillometric method.

Referring to FIG. 6, the pressing cuff 24 is in a non-pressurized state with air being discharged from inside the pressing cuff 24, and the compression cuff 21 is in a pressurized state with air supplied. The compression cuff 21 extends in the circumferential direction of the left wrist 90 and compresses uniformly with respect to the circumferential direction of the left wrist 90 when pressurized by the pump 32. Between the inner circumferential surface of the compression cuff 21 and the left wrist 90, only the electrode group 40E is present. Thus, the blood vessel can be sufficiently closed without other members hindering the compression by the compression cuff 21. Thus, blood pressure measurement via the oscillometric method can be performed with high precision.

The operation of the blood pressure monitor 1 in performing blood pressure measurement via the oscillometric method will be described generally below. Specifically, when the CPU 100 of the blood pressure monitor 1 receives a blood pressure measurement instruction via the operation portion 52, the pump 32 is turned off by the pump drive circuit 320, the valve 33 is opened, and the air inside the compression cuff 21 is discharged. Note that the output value of the pressure sensor 31 at this time is set as a value corresponding to atmospheric pressure.

Then, the CPU 100 closes the valve 33 and drives the pump 32 via the pump drive circuit 320 to supply air to the compression cuff 21. This causes the compression cuff 21 to expand and the cuff pressure to gradually increase. In the process of pressurizing, to calculate the blood pressure value, the CPU 100 monitors the cuff pressure via the pressure sensor 31 and obtains, as a pulse wave signal, a variable component of the arterial volume generated in the radial artery 91 of the left wrist 90.

The CPU 100 attempts to calculate blood pressure values (of systolic blood pressure and diastolic blood pressure) on the basis of the obtained pulse wave signal via the oscillometric method using a known algorithm. If the blood pressure value cannot be calculated due to a lack of data and the cuff pressure is below the maximum pressure (for example, 300 mmHg), the CPU 100 increases the cuff pressure and attempts to again calculate blood pressure values.

In a case where the blood pressure values can be calculated, the CPU 100 stops the pump 32 via the pump drive circuit 320, opens the valve 33, and discharges the air in the compression cuff 21. The CPU 100 displays the blood pressure value measurement results on the display 50 and stores them in the memory 51. Note that the calculation of the blood pressure values is not limited being performed in the pressurizing process and may be performed in the depressurizing process.

### Detailed Method of Calculating Pulse Transit Time

To measure with high precision pulse transit time, i.e., the time difference between the pulse wave signal PS1 and the pulse wave signal PS2, the pulse wave signals PS1, PS2 need to be extracted with high precision. For this, firstly, noise outside of the frequency characterizing the voltage signals v1, v2 (pulse wave signal) must be removed and data with a high S/N ratio (i.e., large dynamic range) must be obtained.

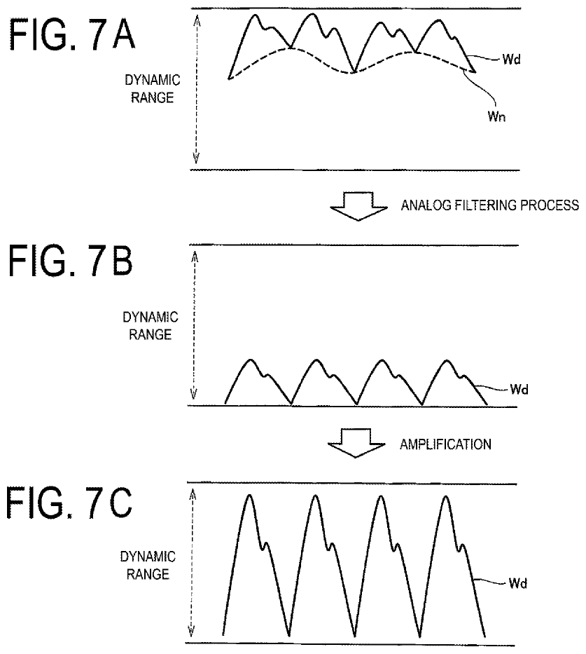

FIGS. 7A to 7C are diagrams for describing the need for an analog filter. FIG. 7A illustrated a case where an unnecessary frequency component (noise wave component Wn) other than the desired frequency component (desired wave component Wd) is superimposed on a voltage signal (analog data) detected by the pair of detection electrodes.

After the analog data is converted to digital data by A/D conversion, the noise wave component Wn can be removed via digital filtering. However, in this case, the dynamic range of the desired wave component Wd is small. Thus, the S/N ratio of the data relating to the desired wave component Wd post digital conversion is low.

Thus, after the noise wave component Wn is removed via analog filter (see FIG. 7B), the dynamic range of the desired wave component Wd is increased by amplifying the desired wave component Wd (see FIG. 7C). By inputting the desired wave component Wd into the CPU 100, the pulse wave signal can be obtained with high precision.

To measure the pulse transit time with high precision, the frequency characteristics (frequency dependence) of the filter must be considered.

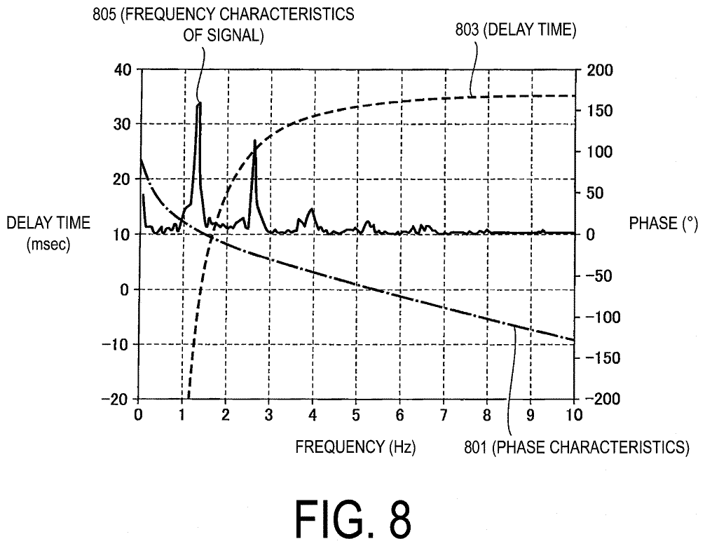

FIG. 8 is a diagram for describing the phase characteristics of the filter. In FIG. 8, the vertical axis on the right indicates the amount of phase change, the vertical axis on the left indicates the delay time, and the horizontal axis indicates the frequency. FIG. 8 illustrates an example where a low pass filter with a cutoff frequency of 10 Hz and a high pass filter with a cutoff frequency of 0.5 Hz is used as an analog filter.

Referring to FIG. 8, a graph 801 shows the frequency characteristics (phase characteristics) of the filter. A graph 803 shows the phase characteristics shown by graph 801 converted into time, i.e., the delay time characteristics. A graph 805 shows the frequency characteristics of a voltage signal (for example, the voltage signal v1), which is a pulse wave signal. In FIG. 8, for example, there is a voltage signal peak at approximately 1.2 Hz, and at this point the amount of phase change is approximately 10°.

In this example, the voltage signals v1, v2 are both pulse wave signals, however, due to the measurement position and the like being different, the frequency components of the waveforms of the voltage signals v1, v2 do not match. Thus, when the voltage signals v1, v2 undergo filtering processing as described above, the amount of phase change in the voltage signal v1 and the amount of phase change in the voltage signal v2 are different. Accordingly, to measure the pulse transit time with high precision, the difference in phase change between the voltage signal v1 and the voltage signal v2 needs to be decreased. The configuration and processing for decreasing the difference in phase change will be described in detail below.

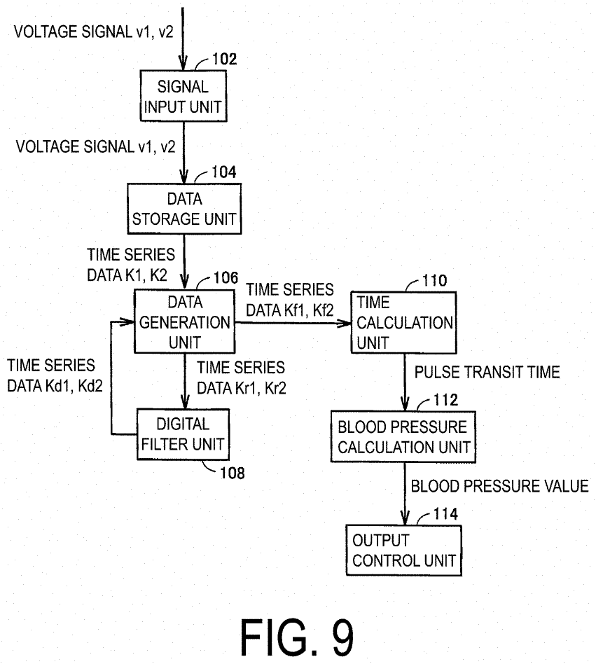

FIG. 9 is a block diagram illustrating the functional configuration of the blood pressure monitor 1. Specifically, FIG. 9 illustrates a functional configuration of the blood pressure monitor 1 used to measure pulse transit time.

Referring the FIG. 9, the blood pressure monitor 1 includes, as a main functional configuration, a signal input unit 102, a data generation unit 106, a digital filter unit 108, a time calculation unit 110, a blood pressure calculation unit 112, and an output control unit 114. Each function is realized, for example, by the CPU 100 of the blood pressure monitor 1 executing a program stored in the memory 51. Note that one or more or all of these functions may be configured to be realized by hardware. The blood pressure monitor 1 further includes a data storage unit 104 realized by the memory 51.

The signal input unit 102 receives input of the voltage signals v1, v2 (digital data) output from the A/D converter 405 for each predetermined sampling period. The signal input unit 102 sequentially stores the received voltage signal v1, v2 in the data storage unit 104.

The data storage unit 104 stores time series data of the voltage signal v1 and time series data of the voltage signal v2. Specifically, the data storage unit 104 stores time series data of the voltage signals v1, v2 from the present time to a predetermined number of previous cycles. For example, a signal value of the voltage signal v1 (digital value of the voltage signal) of the present time is defined as v1(m), the signal value of one sampling period before is defined as v1(m−1), and the signal value of two sampling periods before is defined as v1(m−2). Hereinafter, the signal value of n number of previous sampling periods is defined as v1(m−n).

When the data generation unit 106 and the digital filter unit 108 functioning as a digital signal processing unit use the signal values from the present time to n number of previous sampling periods, time series data including n+1 number of signal values, i.e., v1(m), v1(m−1), v1(m−2), . . . , v1(m−n), is stored in the data storage unit 104. That is, time series data K1 (the signal values v1(m−n) to v1(m)) of the voltage signal v1 are stored. Similarly, time series data K2 (the signal values v2(m−n) to v2(m)) of the voltage signal v2 are stored in the data storage unit 104.

The data generation unit 106 generates time series data Kr1 (the signal values v1(m) to v1 (m−n)), which is the time series data K1 of the voltage signal v1 arranged in reverse chronological order. In a similar manner, the data generation unit 106 generates time series data Kr2 (the signal values v2(m) to v2 (m−n)), which is the time series data K2 of the voltage signal v2 arranged in reverse chronological order. Note that in the case where the time series data K1 and the time series data K2 of a predetermined time period (for example, 5 seconds) are accumulated in the data storage unit 104, the data generation unit 106 performs the generation.

The digital filter unit 108 executes filtering processing on the time series data Kr1, Kr2 via digital filtering with the same transfer function G as the analog filter 403 and generates time series data Kd1 (signal value vd1(m) to vd1(m−n)) and time series data Kd2 (from signal value vd2(m) to vd2(m−n)). The time series data Kd1 and the time series data Kd2 are represented by the following Formula (1) and Formula (2), respectively.

Kd1=Kr1×G (1)

Kd2=Kr2×G (2)

Then, the data generation unit 106 generates time series data Kf1 (signal values vd1(m−n) to vd1 (m)), which is the time series data Kd1 arranged in chronological order. Also, the data generation unit 106 generates time series data Kf2 (signal values vd2(m−n) to vd2 (m)), which is the time series data Kd2 arranged in chronological order.

As described above, in the present embodiment, 1) the time series data Kr1, Kr2, which are the time series data K1, K2 arranged in reverse chronological order, are generated; 2) the time series data Kd1, Kd2 are generated via performing digital filtering processing using the transfer function G (the same transfer function G used with the analog filter 403) on the time series data Kr1, Kr2; and 3) time series data Kf1, Kf2, which are the time series data Kd1, Kd2 rearranged in chronological order, are generated.

By performing the digital filtering processing of 2) described above, approximately the same amount of phase shift is produced as in the filtering processing with the analog filter 403 but in the reverse direction, and the data can be returned to chronological order in 3) described above. As a result, the time series data Kf1, Kf2 is data in which the phase shift produced in the filtering processing with the analog filter 403 is reduced.

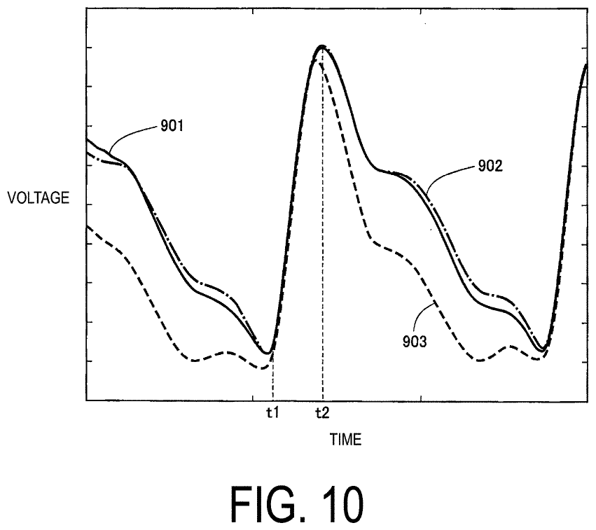

FIG. 10 is a diagram for explaining the advantages of digital signal processing according to the present embodiment. In FIG. 10, the vertical axis indicates voltage and the horizontal axis indicates time. Referring to FIG. 10, a waveform 901 illustrates the waveform of a pulse wave signal (for example, the voltage signal v1) prior to filtering processing by an analog filter. A waveform 902 illustrates the waveform of a pulse wave signal that has been subjected to the digital signal processing described in 1) to 3) above after filtering processing by an analog filter. A waveform 903 illustrates the waveform of a pulse wave signal that has not been subjected to the digital signal processing described in 1) to 3) above and has only been subjected to filtering processing by an analog filter.

As shown in FIG. 10, due to a phase change caused by an analog filter, the waveform 903 is changed a great amount from the waveform 901. On the other hand, the waveform 902 is very similar to the waveform 901, and it can be seen that the amount of phase change due to analog filtering processing is reduced. Specifically, both the waveform 901 and the waveform 902 share a time t1 as a rising time point, and the waveform 901 and the waveform 902 share a time t2 as a peak time point. The waveform 901 and the waveform 903 have their rising time points and peak time points at different times. The rising time point, for example, is a point in time when the instantaneous value (voltage value) of a signal increases as time passes.

Returning to FIG. 9, the time calculation unit 110 calculates the time difference Δt between the pulse wave signal PS1 and the pulse wave signal PS2 as the pulse transit time on the basis of the pulse wave signal PS1 indicated by the time series data Kf1 and the pulse wave signal PS2 indicated by the time series data Kf2.

For example, the time calculation unit 110 calculates the time difference Δt between the point in time of a peak A1 of the pulse wave signal PS1 and the point in time of a peak A2 of the pulse wave signal PS2 as the pulse transit time. Also, the time calculation unit 110 may calculate a time difference Δt1 between the rising time point of the pulse wave signal PS1 and the rising time point of the pulse wave signal PS2 as the pulse transit time. Alternatively, the time calculation unit 110 may calculate an average value of the time difference Δt and the time difference Δt1 as the pulse transit time. In this way, the precision of the pulse transit time can be further increased.

The blood pressure calculation unit 112 calculates a blood pressure value on the basis of the pulse transit time calculated by the time calculation unit 110. Specifically, the blood pressure calculation unit 112 calculates (estimates) a blood pressure value on the basis of the pulse transit time using a preset correspondence formula for pulse transit time and blood pressure value. The preset correspondence formula for pulse transit time and blood pressure value is represented by the following Formula (3), which is, for example, a known fractional function (see JP 10-201724 A). In this formula, DT is pulse transit time, EBP is blood pressure value, and α and β are known coefficients or constants.

EBP=(α/DT²)+β (3)

Note that the correspondence formula is not limited to Formula (3) described above and, for example, a term 1/DT and a term DT may be used in addition to the term 1/DT². Another known correspondence formula may also be used.

The output control unit 114 displays on the display 50 the blood pressure value calculated by the blood pressure calculation unit 112. Also, the output control unit 114 may have a configuration in which the blood pressure value is output as sound via a speaker (not illustrated) provided in the blood pressure monitor 1.

### Processing Procedure of Measuring Blood Pressure Value on the Basis of Pulse Transit Time

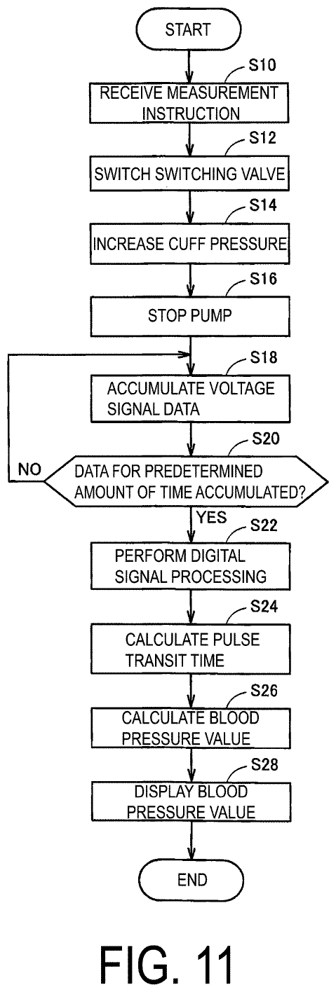

FIG. 11 is a flowchart illustrating a processing procedure of measuring a blood pressure value on the basis of pulse transit time. Referring to FIG. 11, the CPU 100 of the blood pressure monitor 1 receives via the operation portion 52 an instruction to measure blood pressure on the basis of pulse transit time (step S10). The CPU 100 drives the switching valve 35 and switches the connection destination of the pump 32 and the valve 33 to the pressing cuff 24 (step S12).

The CPU 100 inflates the pressing cuff 24 and increase a cuff pressure Pc (step S14). Specifically, the CPU 100 closes the valve 33 and drives the pump 32 via the pump drive circuit 320, increasing the cuff pressure Pc by sending air in the pressing cuff 24. Then, when the cuff pressure Pc reaches a preset pressure, the CPU 100 stops the pump 32 (step S16). In this way, the cuff pressure Pc is set to a preset pressure. In this state, the CPU 100 starts the process of obtaining a pulse transit time as described in the following steps.

Specifically, the CPU 100 receives input of the voltage signals v1, v2 and accumulates in the memory 51 the time series data of each of the voltage signals v1, v2 (step S18). The CPU 100 determines whether the time series data for a predetermined amount of time has been accumulated (step S20). If the time series data for a predetermined amount of time has not been accumulated (NO in step S20), then the CPU 100 executes the process of step S18.

If the time series data for a predetermined amount of time has been accumulated (YES in step S20), then the CPU 100 executes digital signal processing (step S22). Specifically, the CPU 100 generates the time series data Kr1, which is the time series data K1 of the voltage signal v1 arranged in reverse chronological order, and the time series data Kr2, which is the time series data K2 of the voltage signal v2 arranged in reverse chronological order. The CPU 100 generates the time series data Kd1, Kd2, which are the time series data Kr1, Kr2 having undergone digital filtering processing using the transfer function G. The CPU 100 generates the time series data Kf1, Kf2, which are the time series data Kd1, Kd2 arranged in chronological order. In this way, the CPU 100 generates the pulse wave signal PS1 corresponding to the time series data Kf1 and the pulse wave signal PS2 corresponding to the time series data Kf2.

Then, the CPU 100 calculates the time difference Δt between the pulse wave signal PS1 and the pulse wave signal PS2 as the pulse transit time (step S24). The CPU 100 calculates a blood pressure value on the basis of the pulse transit time using a correspondence formula for the pulse transit time and the blood pressure value (for example, Formula (3)) (step S26). The CPU 100 displays the calculates blood pressure value on the display 50 (step S28), and the process ends.

### Advantages

According to the present embodiment, a phase shift in the pulse wave signals due to filtering processing can be reduced. This allows the pulse transit time calculated by comparing the pulse wave signals to be measured with higher precision. As a result, the precision of the blood pressure measurement on the basis of pulse transit time is increased.

Also, according to the present embodiment, the entire waveform of each pulse wave signal can be obtained with high precision. As such, the pulse transit time can be calculated by comparing the entire waveform of one of the pulse wave signals to the entire waveform of the other one of the pulse wave signals (for example, comparing the rising time points, the peak time points, and the like).

### Other Embodiments

1) In the embodiments described above, the pulse wave sensor 401 and the pulse wave sensor 402 was described as having a configuration in which the pulse wave of the artery (radial artery 91) that runs through the target measurement site (the left wrist 90) is detected as a change in impedance. However, the configuration is not limited thereto.

For example, the pulse wave sensors may each include a light emitting element that irradiates light at the artery running through the corresponding portion of the target measurement site and a light receiving element that receives the reflected light (or transmitted light) of the light, the pulse wave of the artery being detected as a change in volume (photoelectric method). Alternatively, the pulse wave sensors may each include a piezoelectric sensor that is in contact with the target measurement site, the strain due to the pressure of the artery running through the corresponding portion of the target measurement site being detected as a change in electric resistance (piezoelectric method). In another alternate, the pulse wave sensors may each include a transmission element that sends an electromagnetic wave (transmission wave) to the artery running through the corresponding portion of the target measurement site and a reception element that receives the reflected wave of the electromagnetic wave, the change in distance between the artery and the sensors due to the pulse wave of the artery being detected as a phase shift between the transmission wave and the reflected wave (electromagnetic wave irradiation method).

2) In the embodiment described above, the belt 20, the pressing cuff 24, and the solid material 22 were given as examples of a pressing portion, however no such limitation is intended. For example, the pulse wave sensor 401 and the pulse wave sensor 402 may be pressing portions that mechanically expand in the thickness direction from the outer circumferential surface of the compression cuff 21 in a non-pressurized state. Also, in the embodiment described above, the pressing cuff 24 fluid bag was given as an example of an expandable member, however no such limitation is intended. For example, the solid material 22 may be pressed against the pulse wave sensor 401 and the pulse wave sensor 402 by an expandable member mechanically expanding in the thickness direction.

3) In the embodiment described above, a configuration was described in which pulse transit time is calculated by comparing two pulse wave signals obtained from two pulse wave sensors, however the configuration is not limited thereto. For example, the pulse transit time may be calculated by comparing the pulse wave signal obtained from one pulse wave sensor (for example, the pulse wave sensor 401 or the pulse wave sensor 402) and an electrocardiographic signal obtained by an electrocardiographic sensor. In this case, analog signal processing and digital signal processing similar to those described above are also performed on the electrocardiographic signal.

The electrocardiographic sensor includes a pair of electrocardiographic electrodes and detects an electrocardiographic signal using the two electrocardiographic electrodes. The electrocardiographic electrodes, for example, can be brought into contact with and attached to the left/right hands or arms of the body. The electrocardiographic electrodes are connected to the voltage detection circuit 49 via a cable. The voltage detection circuit 49 detects an electrocardiographic signal via the cable and outputs the electrocardiographic signal to the CPU 100 via the wire 72. Note that the analog filter for performing filtering processing on the electrocardiographic signal may be the same as or different from the analog filter for performing filtering processing on the pulse wave signal. If a dedicated analog filter is separately prepared for performing filtering processing on the electrocardiographic signal, digital signal processing on the electrocardiographic signal is performed using the same transfer function as the transfer function used for the dedicated analog filter.

Typically, the CPU 100 (time calculation unit 110) calculates the time difference between the rising time point of the pulse wave signal and the peak time point of the electrocardiographic signal as the pulse transit time. However, the CPU 100 may calculate as the pulse transit time the time difference between the peak time point of the pulse wave signal indicated by the time series data post digital signal processing and the peak time point of the electrocardiographic signal indicated by the time series data post digital signal processing.

4) In the embodiment described above, a configuration was described in which the CPU 100 functions as a data generation unit, a digital filter unit, a time calculation unit, a blood pressure calculation unit, and an output control unit, however the configuration is not limited thereto. For example, a computer device (for example, a smart phone or the like) configured to communicate with the blood pressure monitor 1 may sequentially receive via the network 900 the voltage signals v1, v2 (digital data), function as a data generation unit, a digital filter unit, a time calculation unit, a blood pressure calculation unit, and an output control unit and calculate the pulse transit time and the blood pressure value, and display the blood pressure value.

(5) In the embodiments described above, a program may be provided that causes a computer to function and execute controls such as those described in the flowcharts described above. Such a program can also be provided as a program product stored on a non-temporary computer-readable recording medium attached to a computer, such as a flexible disk, a compact disk read only memory (CD), a secondary storage device, a main storage device and a memory card. Alternatively, a program may be provided, which is stored on a recording medium such as a hard disk built into a computer. The program may also be provided by download via a network.

With the program, required modules from among program modules provided as part of the computer operating system (OS) may be called in a predetermined sequence at a predetermined timing to execute processing. In this case, the modules described above are not included in the program itself, and the process is executed in cooperation with the OS. Programs that do not include such modules may also be included in the program according to the present embodiment.

In addition, the program according to the present embodiment may be provided integrated into a part of a different program. In this case as well, the program according to the present embodiment per se does not include the modules included in the different programs described above, and the process is executed in cooperation with the different program. Such a program integrated in a different program shall also be within the scope of the program according to the present embodiment.

The configuration given as an example of the embodiment described above is an example configuration of the present invention. The configuration can be combined with other known technology, and parts thereof may be omitted or modified within the scope of the present invention. Furthermore, the processes and configurations of other embodiments may be employed as appropriate to the embodiments described above.

The embodiments described herein are illustrative in all respects and are not intended as limitations. The scope of the present invention is indicated not by the descriptions above but by the claims and includes all meaning equivalent to the scope and changes within the scope.

### REFERENCE SIGNS LIST

1 Blood pressure monitor
41, 46 Pair of current electrodes
10 Body
10b Bottom surface
15 Buckle
20 Belt
21 Compression cuff
22 Solid material
23 Band
24 Pressing cuff
25, 26 Plate-like member
27, 28 Connecting rod
29 Fixing portion
31, 34 Pressure sensor
32 Pump
33 Valve
35 Switching valve
38a, 38b, 39a, 39b Air line
40 Impedance measurement portion
40E Electrode group
42, 43, 44, 45 Pair of detection electrodes
49 Voltage detection circuit
50 Display
51 Memory
52 Operation portion
53 Battery
59 Communication unit
71, 72 Wire
90 Left wrist
91 Radial artery
100 CPU
102 Signal input unit
104 Data storage unit
106 Data generation unit
108 Digital filter unit
110 Time calculation unit
112 Blood pressure calculation unit
114 Output control unit
10, 340 Oscillation circuit
320 Pump drive circuit
401, 402 Pulse wave sensor
403 Analog filter
404 Amplifier
405 A/D converter
900 Network

1. A measurement device, comprising:
a first sensor configured to detect a first signal indicating a pulse wave of a subject;
a second sensor configured to detect a second signal indicating a pulse wave or an electrocardiogram of the subject;
a first signal processing unit configured to perform filtering processing via an analog filter configured to have a predetermined transfer function on the first signal detected by the first sensor and the second signal detected by the second sensor and converts the first signal detected by the first sensor and the second signal detected by the second sensor to digital data; and
a second signal processing unit configured to perform signal processing including filtering processing via a digital filter on first time series data of the first signal converted to digital data by the first signal processing unit and second time series data of the second signal converted to digital data by the first processing unit; wherein
the second signal processing unit
generates third time series data, the third time series data being the first time series data prior to the filtering processing via the digital filter arranged in reverse chronological order,
generates fourth time series data, the fourth time series data being the second time series data prior to the filtering processing via the digital filter arranged in reverse chronological order,
performs filtering processing via the digital filter configured to have the predetermined transfer function on the third time series data and the fourth time series data,
generates fifth time series data, the fifth time series data being the third time series data post filtering processing by the digital filter arranged in chronological order, and
generates sixth time series data, the sixth time series data being the fourth time series data post filtering processing by the digital filter arranged in chronological order;
the measurement device further comprising
a time calculation unit configured to calculate a pulse transit time on the basis of a signal indicated by the fifth time series data and a signal indicated by the sixth time series data.

2. The measurement device according to claim 1, wherein
the second signal is a signal indicating a pulse wave; and
the first sensor and the second sensor are configured to each detect a pulse wave at a portion of an artery running through a target measurement site of the subject corresponding to where the first sensor and the second sensor are located.

3. The measurement device according to claim 2, wherein
the time calculation unit is configured to
calculate a time difference between a rising time point of the signal indicated by the fifth time series data and a rising time point of the signal indicated by the sixth time series data as a pulse transit time, or
calculate a time difference between a peak time point of the signal indicated by the fifth time series data and a peak time point of the signal indicated by the sixth time series data as a pulse transit time.

4. The measurement device according to claim 1, wherein
the second signal is a signal indicating an electrocardiogram; and
the time calculation unit is configured to calculate a time difference between a rising time point of the signal indicated by the fifth time series data and a peak time point of the signal indicated by the sixth time series data as a pulse transit time.

5. The measurement device according to claim 1, further comprising
a data storage unit configured to store the first time series data and the second time series data, wherein
the second signal processing unit is configured to execute the signal processing when the first time series data and the second time series data for a predetermined amount of time are accumulated in the data storage unit.

6. The measurement device according to claim 1, further comprising
a blood pressure calculation unit configured to calculate a blood pressure value on the basis of a pulse transit time calculated by the time calculation unit.

7. The measurement device according to claim 6, further comprising:
a display; and
a display control unit configured to display a blood pressure value calculated by the blood pressure calculation unit on the display.

8. A measurement method, comprising:
detecting a first signal indicating a pulse wave of a subject;
detecting a second signal indicating a pulse wave or an electrocardiogram of the subject;
performing filtering processing via an analog filter with a predetermined transfer function on the first signal and the second signal and converting the first signal and the second signal to digital data; and
performing signal processing including filtering processing via a digital filter on first time series data of the first signal converted to digital data and second time series data of the second signal converted to digital data, the performing signal processing comprising
generating third time series data, the third time series data being the first time series data of the first signal prior to the filtering processing via the digital filter arranged in reverse chronological order,
generating fourth time series data, the fourth time series data being the second time series data of the second signal prior to the filtering processing via the digital filter arranged in reverse chronological order,
performing filtering processing via the digital filter with the predetermined transfer function on the third time series data and the fourth time series data,
generating fifth time series data, the fifth time series data being the third time series data post filtering processing by the digital filter arranged in chronological order, and
generating sixth time series data, the sixth time series data being the fourth time series data post filtering processing by the digital filter arranged in chronological order;
the measurement method further comprising calculating a pulse transit time on the basis of a signal indicated by the fifth time series data and a signal indicated by the sixth time series data.
